(19) United States
(12) Patent Application Publication
(10) Pub. No.: US 2023/0065235 A1
(43) Pub. Date: Mar. 2, 2023

(54) PRINTABLE CARBON NANOTUBE-BASED CARBON DIOXIDE SENSOR

(71) Applicant: Brewer Science, Inc., Rolla, MO (US)

(72) Inventors: Zun Chen, Springfield, MO (US); Christopher Landorf, Springfield, MO (US); Vijaya Kayastha, Springfield, MO (US)

(21) Appl. No.: 17/745,375

(22) Filed: May 16, 2022

Related U.S. Application Data
(60) Provisional application No. 63/188,518, filed on May 14, 2021.

(51) Int. Cl. G01N 33/00; G01N 27/414

(52) U.S. Cl. CPC G01N 33/004; G01N 27/4146; B82Y 40/00

(57) ABSTRACT

A carbon nanotube sensor device for detecting CO₂ and methods of its production and use. A printable polyethylenimine (PEI)-functionalized carbon nanomaterial paste may be used to form the active sensing layer of the device, which is particularly sensitive to CO₂. A separate printed heating layer may be used to maintain the working temperature of the sensor, as well as to remove and/or clear volatile gases from the sensor.

### CROSS-REFERENCE TO RELATED APPLICATIONS

The present application claims the priority benefit of U.S. Provisional Patent Application Ser. No. 63/188,518, filed May 14, 2021, entitled PRINTABLE CARBON NANOTUBE-BASED CARBON DIOXIDE SENSOR, incorporated by reference in its entirety herein.

### FEDERALLY SPONSORED RESEARCH OR DEVELOPMENT

This invention was made with government support under Federal Award Number W912HZ19C0048, “Concept Printed Sensor Systems for Sensing and Monitoring of Harmful Gases awarded by the U.S. Army Corps of Engineers. The government has certain rights in the invention.

### BACKGROUND OF THE INVENTION

### Field of the Invention

The present invention relates to devices for, and methods of, sensing carbon dioxide (CO₂), as well as PEI-functionalized carbon nanomaterials used for forming the active sensing layer of the devices.

### Description of Related Art

There is a need for carbon dioxide (CO₂) detectors for a variety of applications, such as agricultural, bio-related, food fresh keeping packaging, brewing and carbonated drinks, and dry ice production.

Infrared (IR) technology is predominantly used in commercially available CO₂ detectors. This method uses an IR light source directed at an IR light detector, which detects the CO₂ concentration between the source and sensor by measuring the absorbance of the CO₂ gas present at a characteristic wavelength. Systems using the IR technology are relatively large in size and expensive, and furthermore, they suffer from some reliability limitations. The potential for false positives exists, since the IR spectrum of CO₂ has some similarities to the spectra for both oxygen and nitrous oxide.

Conductometric semiconducting metal oxide CO₂ sensors have also been used and generally include a nonconductive solid substrate, at least one metal-oxide sensing electrode, a reference electrode, and a counter electrode positioned on the substrate. This method has high sensitivity and a rapid response time. However, commercial products utilizing this technology are expensive, non-selective to gas analytes and consume considerable energy due to high operational temperatures.

Certain other electronic sensor structures operate by causing a physical change (i.e. electric polarization, magnetic, structural, morphological, or phase changes) or chemical change (i.e. disassociation, reaction, or compositional change) within an “active” layer that results in a corresponding change in an electrical characteristic such as device resistance, capacitance, or inductance. In sensors, this could be the result of a change in the sensing material (or “active” layer) of a sensor, caused by a reaction to an external stimulus. Examples of such sensing applications are described in detail in U.S. Patent Application Publication No. 2018/0067066, which is incorporated by reference herein in its entirety.

In many existing carbon nanotube (CNT) sensor technologies, the thin-film electrically active CNT layers generally consist of highly electrically conducting grains (along randomly oriented individual CNTs or ropes made of multiple CNTs) separated by insulating regions. The insulating regions bridge the ends or sidewalls of the CNTs and tend to trap charge carriers on the conducting grains. The process of electrical conduction in these thin film devices (within the active layers) is generally a variable range quantum mechanical hopping or tunneling process of charge transport across the insulating regions from two or more conducting regions. The pathway of carrier transport through this electrical layer is through a very large number of such processes that form a percolation network from electrical contact to electrical contact.

In such CNT sensor devices, environmental gases interact with the conduction process through hydrogen or Vander Waals bonding at the interface between the electrical conducting and insulating grains in the thin CNT film. These interactions either tend to increase or decrease the effective carrier trapping potential of the conducting grains. These processes will either raise or lower the electrical conductivity of the CNT layer. Over time, these influences can cause the macroscopic electrical conductivity to drift and generally become unstable as the environment surrounding the films changes. In many cases, this unstable behavior results in poor performance of the device or sensor.

As sensor applications require the diffusion of the environmental constituent in order to operate, it is not possible to simply block the environmental constituent from the active surface. Therefore, for sensor applications, it is necessary to clear the active layer of the device so that there is no environmental constituent in or around the active layer and the electronic behavior of the active layer is stable over time.

For gas sensing, individual, discrete solid-state sensors that can operate in normal atmospheric conditions and can quickly and selectively sense gases, like H₂, CO, CH₄, NO₂, and H₂O, are desired by emerging industries for real-time analysis of environmental air. Such solid-state gas detectors could be used in a variety of applications. Many existing sensors are based on metal and/or metal oxide films, nanoclusters, or nanowires that typically detect gases through physisorption or reversible chemical reactions at temperatures from 100°-400° C. These types of devices have been widely utilized in a configuration utilizing a ceramic tube oven heated by a central tungsten or tantalum filament and coated with the gas-sensitive materials. The resistance of the coating is monitored once the oven reaches thermal equilibrium with the surrounding environment, which may take hours. The resistance of the metal film/metal oxide semiconductor fluctuates with the trace gas of interest.

However, the disadvantages of this gas sensing technology include the lack of gas selectivity and the large power consumption. These devices may also be cross-sensitive to other gases, smoke, and water vapor. As a result, they are best used in a known environment where the gas of interest is generally expected to be present and the cross-sensitive effects are not expected. These devices are signal-enhanced so that they are more sensitive to the gas of interest, but false positive events can result from these cross-sensitivities. Since these devices operate best at temperatures well above room temperature, they tend to expend a significant amount of power, on the order of watts. Thus, additional technologies are being developed for room-temperature operation of these devices, but operation at elevated temperatures will generally enhance the signal. Nevertheless, these solid-state gas sensors have the advantage of having low cost, excellent stability, high sensitivity, and a relatively rapid response once thermal equilibrium is reached.

### SUMMARY OF THE INVENTION

The present invention is broadly concerned with devices for, and methods of, sensing carbon dioxide (CO₂). The devices generally comprise an active sensing layer formed from a printable paste comprising PEI-functionalized carbon nanomaterials. Embodiments of the present invention combine the advantages of printed sensors and PEI-functionalized carbon nanomaterials. For example, the printed sensors described herein are advantageously suitable for low operation temperatures and are easily combined with other humidity, temperature, and gas sensors.

In one embodiment, there is provided a carbon dioxide sensor comprising first and second sensing electrodes formed from electrically conductive material and an active sensing layer formed from polyethyleneimine-functionalized carbon nanomaterials comprising branched polyethyleneimine polymers having a weight average molecular weight of less than about 25,000. The sensing electrodes are spaced apart from one another and positioned on a substrate. The active sensing layer is positioned on the substrate in direct physical contact with at least a portion of the first and second sensing electrodes and is positioned to detect a change in an electrical characteristic in response to a change in carbon dioxide concentration in proximity to the active sensing layer.

In another embodiment, there is provided a sensor array comprising the carbon dioxide sensor above. The first and second sensing electrodes are positioned to detect a change in a first portion of the active sensing layer. A third sensing electrode is positioned to detect a change in an electrical characteristic in response to a change in an analyte concentration in proximity to at least one of: (a) a second portion of the active sensing layer different from the first portion of the active sensing layer; and (b) a second active sensing layer.

In another embodiment, there is provided a method of determining the carbon dioxide concentration of a target gas with the sensor above. The method comprises: (i) contacting the active sensing layer with the target gas; and (ii) measuring an electrical characteristic between the at least first and second electrodes.

In another embodiment, there is provided a method of forming a carbon dioxide sensor. The method comprises dispensing a composition onto a substrate. The composition comprises polyethyleneimine-functionalized carbon nanomaterials dispersed in a liquid medium. The polyethyleneimine-functionalized carbon nanomaterials comprises branched polyethyleneimine polymers having a weight average molecular weight of less than about 25,000.

In another embodiment, there is provided a composition comprising polyethyleneimine-functionalized carbon nanomaterials dispersed in a liquid medium. The polyethyleneimine-functionalized carbon nanomaterials comprise branched polyethyleneimine polymers having a weight average molecular weight of less than about 25,000.

### BRIEF DESCRIPTION OF THE DRAWINGS

Figure (FIG. 1 is a schematic (not to scale) of a CO₂ sensor (transducer) according to one embodiment of the present invention;

FIG. 2 is a side view schematic (not to scale) of a CO₂ sensor (transducer), similar to that shown in FIG. 1, according to one embodiment of the present invention;

FIG. 3 is a set of microscopic photographs comparing raw carbon nanotubes to PEI-functionalized carbon nanotubes;

FIG. 4 is a schematic (not to scale) of a 2-pixel CO₂ sensor with separate printed silver heater, according to one embodiment of the present invention;

FIG. 5 is a schematic (not to scale) of an experimental setup for gas sensing testing using a microflow controller, according to one embodiment of the present invention;

FIG. 6 is a graph showing the sensitivity of a PEI-CNT sensor at different CO₂ concentrations;

FIG. 7 is a schematic (not to scale) of a 2-pixel CO₂ sensor with separate printed CNT heater, according to one embodiment of the present invention;

FIG. 8A is a graph showing the temperature profile of a pulse heated sensor over time;

FIG. 8B is a graph showing the electrical current applied to the pulse heated sensor to provide the temperature profile of FIG. 8A;

FIG. 9 is a graph showing the sensitivity of a PEI-CNT sensor at different CO₂ concentrations;

FIG. 10 is a graph showing the sensitivity of a PEI-CNT sensor at different CO₂ concentrations;

FIG. 11 is a graph showing the sensitivity of a PEI-CNT sensor at different CO₂ concentrations;

FIG. 12 is a schematic (not to scale) of a 4-pixel direct pulse heating CO₂ sensor, according to one embodiment of the present invention;

FIG. 13 is a graph showing the temperature vs. pulse power curve for direct pulse heating;

FIG. 14 is a graph showing the silver DC heater voltage vs. temperature curve;

FIG. 15 is a graph showing CO₂ sensor sensitivities using pulse heating under dry and humid environments;

FIG. 16 is a graph showing CO₂ sensor sensitivities using backside DC silver heaters under dry and humid environments; and

FIG. 17 is a graph showing the response of a CO₂ sensor to 10,000 ppm of CO₂, CO, and H₂ gases.

### DETAILED DESCRIPTION

The present invention is broadly concerned with compositions, CO₂ sensing devices formed using the compositions, and methods of forming and using the CO₂ sensing devices. The compositions are generally in the form of printable polyethylenimine (PEI)-functionalized carbon nanomaterial (e.g., carbon nanotube) based dispersions (or pastes), which can be semi-conductive and particularly suitable for use in CO₂ sensing applications. The PEI-functionalized carbon nanomaterial paste may comprise PEI bonded onto oxidized carbon nanomaterials, which are dispersed in a liquid medium. Such dispersions allow the material to be easily processed and enables the ability to coat or otherwise dispense the paste onto various substrates using common printing techniques. Advantageously, the PEI-functionalized carbon nanomaterial can absorb and desorb CO₂ reversibly, as described below. The reversible interaction (adsorption/desorption) between CO₂ and the PEI-functionalized carbon nanomaterial can induce changes in electrical properties, such as conductivity at a certain temperature.

Polyethylenimine (PEI) (also known as polyaziridine or poly(iminoethylene)) refers to a polymer with repeating units comprising an amine group and two carbon aliphatic CH₂CH₂ spacers, which has the following general structure:

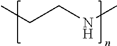

PEI polymers can have different structures and sizes (molecular weights) and can be characterized based on its three types of amine groups: primary, secondary and tertiary. Linear PEI polymers contain all secondary amines, while branched PEI polymers include primary, secondary, and tertiary amino groups. The primary and secondary amino groups can adsorb directly with CO₂ at room temperature to form carbamates. This reaction is reversible, and the desorption reaction can happen at temperatures of between about 25° C. and about 90° C. The mechanism of the absorption/desorption of CO₂ in PEI is shown below:

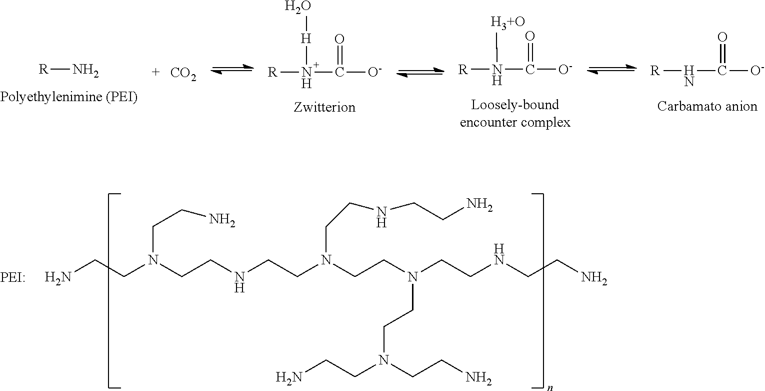

The PEI polymers used in conjunction with the PEI-functionalized carbon nanomaterial compositions and sensing layers of the present invention may have a variety of structures and sizes (molecular weights). However, in certain preferred embodiments, the PEI comprises branched PEI and/or has a weight average molecular weight of about 100 to about 100,000. In certain embodiments, the PEI may have a weight average molecular weight of less than about 25,000, less than about 20,000, less than about 15,000, less than about 10,000, or less than about 5,000. In particularly preferred embodiments, the PEI may have a weight average molecular weight of less than about 1,500, less than about 1,400, less than about 1,300, less than about 1,200, less than about 1,100, less than about 1,000, or less than about 900. In certain embodiments, the PEI may have a weight average molecular weight of about 200 to about 25,000, from about 300 to about 10,000, from about 400 to about 5,000, from about 500 to about 1,500, from about 600 to about 1,300, or from about 700 to about 1,000.

In certain embodiments, a heating layer may be used in conjunction with the PEI-functionalized carbon nanomaterial active sensing layer to maintain the operational temperature of the sensor, as well as to remove/clear analytes remaining from past exposure to environmental and volatile gases from the sensor. By controlling the electrical conduction network of the heating layer, the temperature of the sensing layer can be maintained to enable the interaction (adsorption/desorption) between the CO₂ and the PEI-functionalized carbon nanomaterial sensing layer. The incorporation of a backside heating layer can heat the electrically active sensing layer directly, resulting in the desorption between the environmental gas and the sensing layer, so that the effects of the environment on the electrical conductivity of the films can be eliminated, and the device is refreshable. In some embodiments, the sensing layer may function as both a CO₂ sensing element as well as a heating element. In such embodiments, the sensing layer may be heated to a desired temperature using high-energy AC pulses, while resistance to analyte exposure is measured.

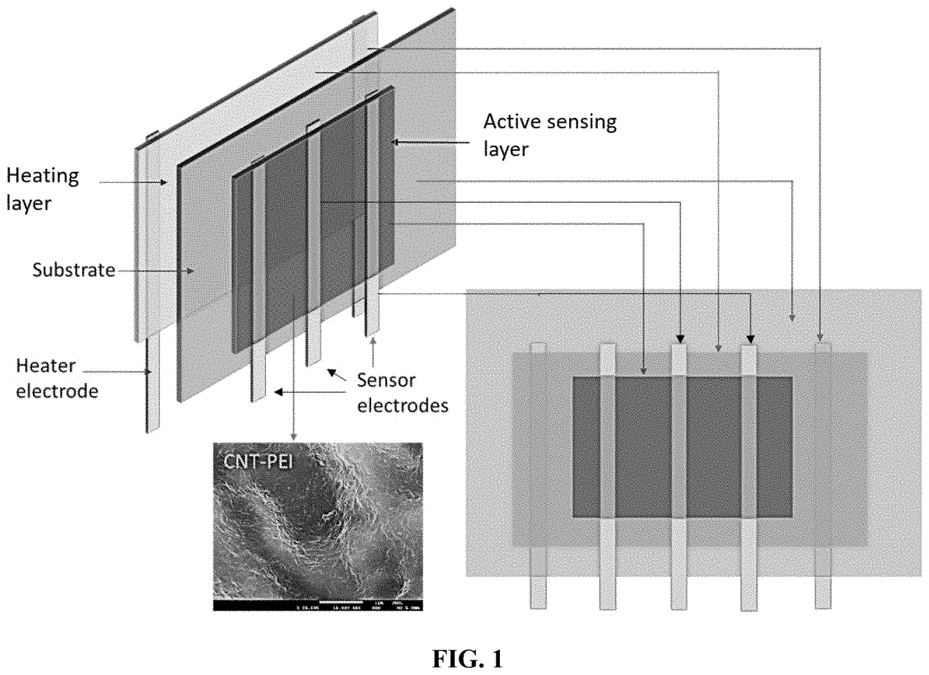

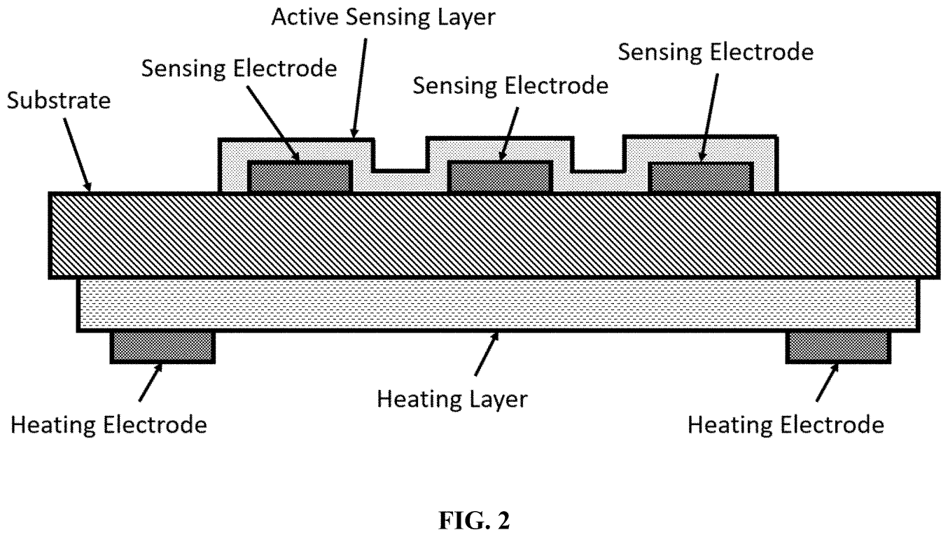

Referring now to FIG. 1, a schematic of exemplary transducer according to one embodiment of the present invention is shown. The transducer generally comprises a substrate, at least two sensing electrodes, and an active sensing layer. Although the embodiment in FIG. 1 shows PEI-functionalized carbon nanotubes (PEI/CNT) as the active sensing layer, it will be understood that other types of PEI-functionalized carbon nanomaterials may also be used in accordance with other embodiments of the present invention. As best shown FIG. 2, the active sensing layer is preferably in direct contact (i.e., physical contact) with each sensing electrode. The transducer may also comprise a separate heating layer and heating electrodes, which may be positioned on the opposite side of the substrate from the active sensing layer and accompanying sensing layer electrodes, as illustrated in FIG. 2. Although not shown, optional layers may also be present on top of, or between, the layers of the transducer described above and may include, but are not limited to, signal enhancement layer(s) and/or filter layer(s) (e.g., between the active sensing layer and the environment) and isolation layer(s) and/or filter layer(s) (e.g., between the active sensing layer and the substrate). Exemplary optional layers and arrangements are described in U.S. Pat. No. 10,352,726, which is incorporated by reference herein in its entirety.

### Substrate

As best shown in FIG. 2, the transducer may comprise a substrate, upon which the other layers (e.g., electrodes, sensing layers, heating layers, etc.) may be printed or otherwise applied. The substrate may be made from any number of materials. In certain embodiments, the substrate is made of, or otherwise comprises, a material selected from the group consisting of polymers, ceramics, metals, single crystal (monocrystalline) materials, and combinations thereof. In certain embodiments, the substrate is made of, or otherwise comprises, an organic polymer or plastic selected from the group consisting of polyimides (such as Kapton® film), polyamides, polysulfones, poly ether sulfones, polyether ether ketone (PEEK), polyethylene terpthalate (PET), polytetrafluoroethylene (PTFE, such as Teflon®), acrylates, methacrylates, styrenics, cycloolefin polymers (such as Zeonor®), cycloolefin copolymers, polyesters, polyethylene naphthalates, and combinations thereof. In certain embodiments, the substrate may preferably have low thermal conductivity. For example, in certain embodiments, the substrate material has a thermal conductivity of from about 0.005 W/m-K to about 1.0 W/m-K, preferably about 0.01 W/m-K to about 0.1 W/m-K. In the same or other embodiments, the substrate may preferably have high thermal stability. For example, in certain embodiments, the substrate material does not degrade, shrink, crack, or decompose at temperatures of preferably up to about 250° C., preferably up to about 150° C. In certain embodiments, the substrate material preferably does not experience hygroscopic expansion or similar deformation, particularly under processing and/or environmental conditions.

In certain embodiments, the thickness of substrate may be from about 2.5 μm to about 250 μm, preferably from about 75 μm to about 200 μm, or more preferably from about 110 μm to about 150 μm. In certain embodiments, the width of the substrate may be from about 0.5 mm to about 100 mm, preferably from about 10 mm to about 20 mm, or more preferably about 10 mm. In certain embodiments, the length of the substrate may be from about 0.5 mm to about 100 mm, preferably from about 10 mm to about 20 mm, or more preferably about 10 mm. It will be appreciated that in one embodiment, multiple sensors may be formed on a larger substrate and then singulated into individual substrates. In the case of that embodiment, the dimensions given above refer to a singulated substrate.

### Sensing Electrodes

Referring again to FIGS. 1 and 2, the transducer may comprise two or more (three shown) sensor electrodes adjacent the substrate. In certain embodiments, the electrodes may be planar electrodes (as shown), although interdigitated electrodes may also be used. In certain preferred embodiments, the electrodes may have high electron or hole mobilities and/or large carrier concentrations. The electrodes may comprise a variety of conductive materials. For example, the electrodes may be formed of, or otherwise comprise, a conductive material selected from the group consisting of silver, poly(3,4-ethylenedioxythiophene) (PEDOT), gold, highly-doped silicon, conductive carbon nanotubes (CNTs), graphene (e.g., graphene-based inks), palladium, copper, aluminum, conductive polymer(s), CNT/graphene-conductive polymer composites, and combinations thereof. In certain preferred embodiments, the electrode material(s) have a low Schottky barrier and low contact resistance to the active sensing layer.

The electrodes may be formed on the substrate (or other layer(s)) by any suitable technique. For example, in certain embodiments, the electrodes may be formed by an application method selected from the group consisting of screen printing, spray coating, Aerosol Jet® printing, ink-jet printing, flexographic printing, gravure printing, lithographic techniques, spin coating, evaporation, sputtering, laser ablation, and combinations thereof.

In certain embodiments, the thickness of one or more, or each, electrode may be from about 0.5 μm to about 50 μm, preferably from about 0.5 μm to about 5 μm, or more preferably from about 1 μm to about 2.5 μm. In certain embodiments, the width of one or more, or each, electrode may be from about 50 μm to about 10 mm, preferably from about 100 μm to about 5 mm, or more preferably about 0.5 mm. In certain embodiments, and particularly when the electrodes are planar, the length of one or more, or each electrode may be from about 1 mm to about 20 mm, preferably from about 2 mm to about 10 mm, or more preferably from about 3 mm to about 5 mm. In certain embodiments, when two (or more) electrodes are used, the distance between the two (or more) electrodes may be from about 10 μm to about 20 mm, preferably from about 0.1 mm to about 10 mm, or more preferably from about 0.5 mm to about 1.5 mm.

### Active Sensing Layer

Referring again to FIGS. 1 and 2, the transducer may comprise an active sensing layer positioned over the sensing electrodes and the substrate. The active sensing layer is operable to provide an electronic signal that changes with a change in property of the active sensing layer. For example, the change in property may be a change in the electrical resistance across the active sensing layer. The change in electrical resistance may be the result of, for example, a change in its electronic structure, defect state, or electronic carrier density. In certain embodiments, the sheet resistance of the active sensing layer may be from about 1 kΩ to about 1 MΩ, preferably from about 1 kΩ to about 150 kΩ, and more preferably from about 30 kΩ to about 90 kΩ. In certain embodiments, upon exposure to CO₂, the resistance of the active sensing layer can change proportionally to the change in the concentration of the CO₂ that is exposed to the active sensing layer. For example, in certain embodiments, the change in resistance should result in an output signal change of at least about 0.5% per 100 ppm change, and preferably at least about 5% per 100 ppm change.

In certain preferred embodiments, the material used to form the active sensing layer may be a planar material that can be provided as a film. In certain embodiments, the active sensing layer may have an average thickness that is about 1 nm to 5000 nm, preferably about 2 nm to about 1000 nm, more preferably from about 3 nm to about 50 nm. In particularly preferred embodiments, the active sensing layer may have an average thickness of less than about 300 nm. As explained below, in certain embodiments, the active sensing layer may have varying topography (e.g., in conformally coated layers) and/or thickness (e.g., in flat-topped encasing layers) across the entirely of the active sensing layer. However, regardless the geometry of the active sensing layer, it should be understood that the average thicknesses described herein refer to the average distance between the exposed surface of the active sensing layer and the underlying substrate or electrode (or other optional layer) upon which the active sensing layer resides. In certain embodiments, the width of the active sensing layer may be from about 0.1 mm to about 20 mm, or preferably from about 0.5 mm to about 5.0 mm. In certain embodiments, the length of the active sensing layer may be from about 0.5 mm to about 20 mm, or preferably from about 1 mm to about 10 mm.

The active sensing layer may be deposited by any suitable technique, including those selected from the group consisting of screen printing, spray coating, Aerosol Jet® printing, ink-jet printing, dip coating, airbrush techniques, flexographic printing, gravure printing, lithographic techniques, spin coating, lamination, and combinations thereof. It will be appreciated that the active sensing layer shown in FIG. 2 is representative only and may not be shown to scale. Thus, it will be further appreciated that, depending on the active sensing layer thickness and deposition technique, the active sensing layer may have substantially constant thickness across the layer, or the thickness could be varied. For example, in certain embodiments, a relatively thinner active sensing layer may be conformally coated over the substrate and electrodes as shown in FIG. 2. Alternatively, in certain embodiments, a relatively thicker active sensing layer may completely encase the electrodes and therefore have varying thickness across the layer (i.e., thinner at the portions covering the electrodes and thicker portions in areas not covering the electrodes).

In particularly preferred embodiments, the active sensing layer comprises polyethylenimine-functionalized carbon nanomaterials. For example, as described above, the active sensing layer may be formed from a printable, polyethylenimine (PEI)-functionalized carbon nanomaterial (e.g., carbon nanotube) composition, which may be in the form of a dispersion (or paste).

In certain embodiments, the conductive PEI-functionalized carbon nanomaterial paste may be produced by first dispersing conductive carbon nanomaterials (non-PEI-functionalized) in a liquid medium (i.e., dispersion medium), such as a solvent, to form a carbon nanomaterial dispersion. Suitable carbon nanomaterials include, but are not limited to, oxidized (such as those described in U.S. Pat. No. 9,157,003, which is incorporated by reference in its entirety herein) or non-oxidized carbon black, carbon nanotubes, graphene, carbon nanohorns, carbon nanodiscs, fullerene, their derivatives, and mixtures thereof. In certain preferred embodiments, the carbon nanomaterial comprises oxidized carbon nanotubes. In certain embodiments, the liquid medium may comprise a solvent selected from the group consisting of water, alcohols (such as isopropanol), diols (such as 1,2-propanediol and 2-methyl-1,3-propanediol), and polar water-miscible solvents (such as acetone, n-methyl-2-pyrrolidone [NMP], and 1,3-dimethyl-2-imidazolidinone [DMI]), and combinations thereof. In certain preferred embodiments, the solvent comprises 2-methyl-1,3-propanediol.

In certain embodiments, the carbon nanomaterial dispersion may comprise from about 0.001% to about 1%, preferably from about 0.05% to about 0.5%, by weight of the carbon nanomaterials, with the total weight of the dispersion taken as 100% by weight. In certain embodiments, the carbon nanomaterial dispersion may comprise from about 99% to about 99.999%, more preferably from about 99.5% to about 99.95%, by weight of the solvent, based upon the total weight of the dispersion taken as 100% by weight.

Once the (non-PEI-functionalized) carbon nanomaterials dispersion is prepared, the PEI-functionalized carbon nanomaterial dispersion (or paste) can be formed by dispersing PEI into the carbon nanomaterial dispersion. In one embodiment, the PEI is dispersed or dissolved in water and then the PEI-water dispersion is mixed with the carbon nanomaterials dispersion. In the embodiment where a PEI-water dispersion is mixed with the carbon nanomaterials dispersion, the PEI is present in the PEI-water dispersion in an amount of preferably from about 10% by weight solids to about 30% by weight solids as a percentage of the PEI-water dispersion taken as 100% by weight.

As described above, the PEI may have a variety of polymer structures and molecular weights. However, it was surprisingly discovered that the use of relatively lower molecular weight PEI can impart improved CO₂ sensitivity to the sensor, and particularly to the active sensing layer. Therefore, in certain preferred embodiments, the PEI may have a weight average molecular weight of less than about 25,000, less than about 5,000, less than about 3,000, less than about 1,500, less than about 1,400, less than about 1,300, less than about 1,200, less than about 1,100, less than about 1,000, or less than about 900.

The PEI can generally be dispersed in the presence of suitable solvent systems, including those of the carbon nanomaterials dispersions described above, to form a substantially homogeneous dispersion or paste. During this process, the PEI may chemically bond to acid functional group(s) on the carbon nanomaterials, or the PEI may wrap around the carbon nanomaterials. In certain preferred embodiments, the PEI is chemically bonded to the surface of oxidized carbon nanomaterials. The PEI can be mixed or otherwise dispersed into the carbon nanomaterial paste using a variety of methods. However, in certain preferred embodiments, the carbon nanomaterial dispersion and amine-polymer (PEI) can be mixed by a probe ultrasonicator or a 3-roll mill to provide sufficient mixing.

The level of PEI functionalization can depend on factors such as the types of PEI polymer and carbon nanomaterials used. However, in certain embodiments, the carbon nanomaterial within the PEI-functionalized carbon nanomaterial paste can comprise from about 80% to about 99.9% by weight, preferably from about 90% to about 99% by weight, based on the total weight of the PEI-functionalized nanomaterials within the paste taken as 100% by weight (i.e., on a dry basis). In certain embodiments, the polymer content (PEI) within the PEI-functionalized carbon nanomaterial paste can comprise from about 0.1% to about 20% by weight, preferably from about 0.5% to about 15% by weight, and more preferably from about 3% to about 10% by weight, based on the total weight of the PEI-functionalized nanomaterials within the paste taken as 100% by weight (i.e., on a dry basis).

In certain embodiments, one or more optional components may also be mixed with the PEI and carbon nanomaterials to form the active sensing layer material. For example, a signal enhancement material, such 2-hydroxyethyl cellulose, may be mixed with the PEI and/or carbon nanomaterials dispersion or otherwise incorporated into the final paste composition.

The resulting PEI-functionalized carbon nanomaterial paste can advantageously be easily processed and dispensed, for example by screen printing or other suitable printing process, onto the electrodes, substrate, and/or other layer(s) as appropriate. In certain embodiments, after the PEI-functionalized carbon nanomaterial paste is deposited onto the electrodes, substrate, and/or other layer(s) as appropriate, the paste is preferably then baked to remove solvents at a temperature of from about 40° C. to about 80° C., more preferably from about 50° C. to about 60° C., preferably in a vacuum oven at 30″Hg.

In certain embodiments, and particularly when two or more electrodes are present, the printed active sensing layer should be touching (i.e., direct physical contact) with two or more of the electrodes. For example, as best shown in FIG. 2, in certain embodiments, the active sensing layer is in direct physical contact with each electrode at the sidewalls, upper surfaces, and/or lower surfaces of the electrodes.

### Heating Layer

Referring again to FIGS. 1 and 2, the transducer may comprise a heating layer, which may be formed on the opposite side of the substrate from the active sensing layer (as shown), or on the same side of the substrate as the active sensing layer and sensing electrodes and separated by a dielectric layer (not shown). The heater layer may be configured to maintain the active sensing layer at an appropriate operational temperature. In certain embodiments, the heating layer may also be used to temporarily heat the active sensing layer above the normal operational temperature to clear interfering gasses from the sensing environment. In one embodiment, the heating layer may not be in direct contact with the substrate, and the heating layer may heat the transducer, for example, by radiant or inductive heating.

Suitable materials for the heating layer may include, but are not limited to, metal materials like silver, gold, palladium, copper, aluminum, any conductive metals; conductive polymers; conductive carbon materials, including conductive carbon nanotubes (CNTs), graphene, and carbon black composites; hybrid composites of metals, conductive polymers, and/or conductive carbon materials; and other hybrid materials like highly doped silicon. In certain embodiments, the heating layer material can be a printable conductive ink. In certain preferred embodiments, the heating layer material is a printable silver ink or printable carbon material (e.g., CNT) ink. The heating layer may be deposited by any suitable technique, including those selected from the group consisting of screen printing, spray coating, Aerosol Jet® printing, ink-jet printing, dip coating, airbrush techniques, flexographic printing, gravure printing, lithographic techniques, spin coating, evaporation, sputtering, lamination, ALD, CVD, and PECVD.

In certain embodiments, the heating layer has an average thickness from about 10 nm to 20 μm, preferably from about 50 nm to about 10 μm, and more preferably from about 100 nm to about 5 μm. The thickness may depend upon whether the heater material is a carbon-based or metal-based material. In certain embodiments, the width of the heating layer can be from about 0.1 mm to about 20 mm, or preferably from about 0.5 mm to about 5.0 mm. In certain embodiments, the length of the heating layer can be from about 0.5 mm to about 20.0 mm, or preferably from about 1.0 mm to about 10.0 mm. In certain preferred embodiments, the width and/or length of the heating layer are equal to or greater than the width and/or length of the active sensing layer.

In certain embodiments, and particularly when a silver heating layer is used, the heating layer resistance can be from about 10Ω to about 500Ω, preferably from about 50Ω to about 150Ω, or more preferably from about 80Ω to about 110Ω.

In certain embodiments, and particularly when a printed CNT heating layer is used, the heating layer resistance can be from about 2 kΩ to about 200 kΩ, preferably from about 2 kΩ to about 100 kΩ, or more preferably from about 2 kΩ to about 60 kΩ.

It will be appreciated that the actual structure of the heating element can vary, depending, for example, upon whether the heating layer is a CNT pulse heater or a DC metal heater. The actual construction is largely dictated by the material resistance and the desired heater resistance. For a CNT pulse heater, for example, the heating layer may preferably be a continuous film. For a metal DC heater, however, the heating “layer” may comprise metal traces with a trace width of preferably from about 50 μm to about 2000 μm, or more preferably from about 100 μm to about 1000 μm. Although described herein as separate components, in certain embodiments, and particularly when a metal DC heater is used, the heating layer and heating layer electrodes may be combined (as a unitary construct).

### Heating Layer Electrodes

Referring again to FIGS. 1 and 2, the heating layer electrodes may be incorporated into the sensor structure, which may be positioned over the heating layer (as shown) or under the heating layer, provided contact is still achieved. As noted above, depending on the heating layer structure, the heating layer and electrodes could be one piece (unitary) construction.

In certain embodiments, the electrodes may be planar electrodes (as shown in FIG. 1), although interdigitated electrodes may also be used. In certain preferred embodiments, the heating layer electrodes may have high electron or hole mobilities and/or large carrier concentrations. The heating layer electrodes may comprise a variety of conductive materials, which may be the same or different from the sensing electrodes described above. In certain embodiments, the heating layer electrodes may be formed of, or otherwise comprise, a conductive material selected from the group consisting of silver, poly(3,4-ethylenedioxythiophene) (PEDOT), gold, highly-doped silicon, conductive carbon nanotubes (CNTs), graphene (e.g., graphene-based inks), palladium, copper, aluminum, conductive polymer(s), CNT/graphene-conductive polymer composites, and combinations thereof. In certain preferred embodiments, the electrode material(s) have a low Schottky barrier and low contact resistance to the active sensing layer.

The heating layer electrodes may be formed on the substrate (or other layer(s)) by any suitable technique. For example, in certain embodiments, the heating layer electrodes may be formed by an application method selected from the group consisting of screen printing, spray coating, Aerosol Jet® printing, ink-jet printing, flexographic printing, gravure printing, lithographic techniques, spin coating, evaporation, sputtering, laser ablation, and combinations thereof.

In certain embodiments, the thickness of one or more, or each, heating layer electrode may be from about 0.5 μm to about 50 μm, more preferably from about 0.5 μm to about 5 μm, even more preferably from about 1 μm to about 2.5 μm. In certain embodiments, the width of one or more, or each, heating layer electrode may be from about 50 μm to about 10 mm, preferably from about 100 μm to about 5 mm, or more preferably about 0.5 mm. In certain embodiments, and particularly when the electrodes are planar, the length of one or more, or each electrode may be from about 1 mm to about 20 mm, preferably from about 2 mm to about 10 mm, or more preferably from about 3 mm to about 5 mm. In certain embodiments, when two (or more) electrodes are used, the distance between the two (or more) electrodes may be from about 10 μm to about 20 mm, preferably from about 0.1 mm to about 10 mm, or more preferably from about 2 mm to about 4 mm. In certain embodiments, the heating layer and heating layer electrodes are a single (unitary) structure, and the heating layer/heating layer electrode has a length of about 4 mm and a width of about 4 mm. In certain preferred embodiments, the width and length of the heating layer electrodes are equal to or greater than the width and length of the sensing electrodes.

In certain embodiments, the heating layer and/or heating layer electrodes can be encapsulated with a high-temperature stable insulating layer (for example, a highly porous PTFE membrane) to enhance lifetime of the materials and to prevent exposure to environmental conditions such as moisture or solvents.

### Method of Use

During operation of the sensor, an environmental constituent dispersed in a target gas, such as carbon dioxide, that collects in and around the active sensing layer will diffuse into the sensor structure from the immediate atmospheric environment surrounding the device. As the device comes to equilibrium, the environmental constituent diffuses into the structure, causing changes in the electrical characteristics of the active layer. For both analytes of interest as well as those not of interest, these changes can appear as hysteresis or instabilities in the device electrical conductivity.

Carbon dioxide sensors, or transducers, in accordance with embodiments of the present invention therefore may comprise a pixel sensor that includes at least two electrodes and a conductive active sensing layer. The electronic properties of the active sensing layer can be affected by interactions with environmental stimuli, such as gases in the air (e.g., CO₂). In certain preferred embodiments, this results in a change in resistance between the sensing electrodes as a current passes through the active sensing layer, referred to as “interaction resistance.” Measurement of the interaction resistance using a low current density can be indicative of the type and amount of stimulus that is present in or on the active sensing layer.

Temperature control of the active sensing layer can be important in distinguishing between different environmental constituents that have diffused into the active sensing layer and the surrounding region. By varying the temperature of the sensor pixel, for example, by using the heating layer, the interaction of the sensor with environmental stimuli can be changed, which will change the electronic properties of the active sensing layer, resulting in a different interaction resistance.

Advantageously, multiple pixels can be used together as an array comprising a plurality of pixel sensors. In certain embodiments, each pixel has the same composition. When used with a substrate having a low thermal conductivity, the temperature of each pixel can be controlled independently, and the interaction resistance at various temperatures can be measured simultaneously. This can result in faster sensor speeds, since a range of temperatures can be measured at once, rather than ramping a single sensor pixel through different temperatures.

In certain embodiments, one or more pixels can have different compositions. That is, the pixels can be “mixed and matched,” so that some pixels have active sensing layers formed from a single, uniform composition (i.e., the same composition chemically throughout the layer), while some pixels could have active sensing layers that are a mixture of different compositions (e.g., a signal enhancement material intermixed with another material, such as carbon nanotubes). A signal enhancement layer could be avoided in each pixel, or it could be included in one or more (or even every) pixel of the particular sensor array. In such embodiments, each pixel may be controlled to the same temperature, or to different temperatures. Pixels of different compositions will interact with various gases differently, and can be used to determine the identity and concentration of one gas more precisely, or can determine the identity and concentration of more than one gas simultaneously.

In particular embodiments, for example, a sensor array may comprise first and second sensing electrodes positioned to detect a change in a first portion of an active sensing layer and a third sensing electrode positioned to detect a change in an electrical characteristic in response to a change in an analyte concentration in proximity to a different portion of the sensor. The different portion of the sensor may be, for example, a second portion of the same active sensing layer and/or a second active sensing layer.

Absorption, adsorption, or reaction of various gases with a material will change the resistance of that material as a function of concentration, as well as a function of temperature. Temperature control is tied to sensor precision by the gas absorption curve properties as a function of temperature. If the absorption curve is rapidly changing in the vicinity of the device temperature, control must be maintained so that fluctuations in background temperature do not cause false positive signals. Therefore, additional temperature and relative humidity sensors may be included to control the baseline of the sensor system. If the background temperature and gas absorption curve are well known, an adjusted energy can be dissipated in the heater to achieve the desired temperature.

### Pulsed Heating

In certain embodiments, the transducer may comprise a heating layer, which may preferably be on the side of the substrate that is opposite of the active sensing layer and sensing electrodes. The heating layer electrodes may be connected to pulse heating electronics as described in U.S. Patent Application Publication No. U.S. 2018/0067066, which is incorporated by reference herein in its entirety. The heating electrodes may be pinned on electrode pads, which can be inserted into the female connector of a printed circuit board (PCB) that is connected to the heating electronics via cables.

In certain embodiments, the heating layer can be heated by application of a series of electrical pulses. In certain embodiments, each individual pulse has a duration less than about 100 microseconds, or preferably less than about 70 microseconds. In certain embodiments, each individual pulse has a duration of from about 1 microsecond to about 70 microseconds. A series of pulses can be delivered to the heating layer for a duration of time in order to heat the layer via Joule or ohmic heating. This series of pulses preferably has a short duration. In certain embodiments, the series of pulses can have a duration of less than about 5 seconds, preferably less than about 3 seconds, or more preferably less than about 1 second. In certain embodiments, the series of pulses can have a duration of from about 1 microsecond to about 1 second.

In certain embodiments, the voltage used for the electrical pulses can be from about 25 V to about 500 V, preferably from about 50 V to about 350 V, or more preferably from about 100 V to about 200 V. The electrical carrier current density in the heating layer can be varied depending upon the resistance and desired temperature range. However, in certain embodiments, the current density can be from about 1,000 A/cm² to about 10,000 A/cm², preferably from about 1,500 A/cm² to about 7,000 A/cm², or more preferably from about 2,000 A/cm² to about 5,000 A/cm². The active sensing layer or heating layer experiences an increase in temperature of at least about 50° C., preferably at least about 100° C., more preferably at least about 150° C. In certain embodiments, the increase in temperature may be even higher, for example, from about 200° C. to about 300° C.

In certain embodiments, such as direct pulse heating, the sensor pixel may be heated to a desired temperature by using high energy AC pulses of a pulse power which is a function of pulse width, duration, and pulse AC frequency, applied directly through the sensing electrodes into the active sensing layer. Direct pulse heating provides a relatively simpler sensor design. In one embodiment, four CNT/PEI sensing pixels sharing a common ground can be pulse heated at same or different temperatures using pulse heating electronics that also have data acquisition capability. The temperature in the sensing pixels is controlled by pulse power.

### Direct Current Heating

When a separate DC heater is used, a commercial DC power supply source may be used. In such embodiments, the power supply should generally be capable of providing a DC voltage in the range 0.5 V to 5 V. It will be appreciated that the exact specifications of the DC power supply depend upon the heater material, heater resistance, and design of the sensor.

In the separate heater embodiments, sensing pixels may be heated to desired temperature using separate printed heater, either by pulse heating a CNT heater element or by DC heating of a conductive heater. In certain embodiments, the heater may be printed on the side of the substrate opposite the active sensing layer and sensing electrodes. However, in certain embodiments, the heater may be printed same side of the substrate as the active sensing layer and sensing electrodes, but separated by a dielectric layer. Both CNT pulse heater and conductive DC heater can be encapsulated with a printed, high-temperature stable insulating layer (for example, a polyimide-based encapsulant) to enhance lifetime and to prevent exposure from environmental conditions such as moisture, solvents, etc. The DC voltage required to get a desired temperature of a particularly preferred heater is calculated by following equation:

$$V = \frac{T + 30.82}{43.182}$$

### Sensing

The sensing electrodes may be connected to electronics configured to measure the resistance, impedance, current, voltage, or other changes in the electrical characteristics in the active sensing layer. When exposed to a target gas containing CO₂, the active sensing layer will absorb the CO₂, and form a carbamato anion when the active sensing layer is held at the operational temperature. Preferred operational temperatures are from about 25° C. to about 85° C., more preferably from about 40° C. to about 70° C., even more preferably from about 50° C. to about 55° C. When the CO₂ concentration changes, the amount of formed carbamato anion will change, and the electrical characteristics of the sensing electrodes will change, for example, the resistance of sensing layer changes proportionally to the changing CO₂ concentration. The change in electrical characteristics of the sensing electrodes can then be correlated experimentally to the concentration of CO₂ under known conditions.

In certain embodiments, these measurements may be performed by a multimeter and/or computer, such as a digital multimeter (Flukemeter 8845A) to record resistance data output. The multiplexer in the pulse electronics can be used to take resistance data from multi-sensing pixels.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth compositions, devices, and methods in accordance with embodiments of the invention. It is to be understood, however, that these examples are provided by way of illustration, and nothing therein should be taken as a limitation upon the overall scope of the invention.

### Example 1

### Preparation of Oxidized CNT Paste (Dispersion)

10.123 grams of carbon nanotubes (Tuball, OCSiAl) were placed in a 1-pint polypropylene jar followed by 50 ml of deionized (DI) water. The jar was shaken to wet the tubes. The wet tubes were placed in a 500-ml stainless steel beaker for sonication. About 190 ml of DI water was used to rinse all of the tubes in the sonication vessel. This was sonicated in ice bath using a 1″ probe at 90% power with the booster for 20 minutes (Sonics & Materials, Inc., VCX 750 ultrasonic processor, CV00334 Converter, BHNVC21 Booster, 630-0209 1″ solid probe). Another 340 ml of DI water was added to a 2-L jacketed reactor with oil circulator for heating. 160 ml of nitric acid (68-70%, ARISTAR® ACS, VWR Chemicals BDH, Batavia, Ill.) was added to the reactor. The sonicated CNT slurry was added to the reactor. 860 ml of DI water was measured out and used to wash any remaining tubes from the sonicator into the reactor. The reaction was allowed to proceed for 4 hours. Heating oil was set at 160° C. Reflux condenser chiller was set at 5° C. The reaction product was collected and weighed 1645.5 grams. The reaction product was filtered through an Advantec 3.0 PTFE filter (King Filtration Technologies, Inc., Product: T300A185C). Vacuum timer was set to 1 hour for each wash. Solid was removed, and this was mixed with 1 liter of DI water by hand. This was filtered through a fresh Advantec 3.0 PTFE filter. The material was collected into a HDPE jar (tare weight 27.2 grams). The filtered material was added (89.5 grams including the tare weight). This was frozen overnight and placed in the freeze drier for 2 days. Weight of dried tubes in the jar was 35.3 grams. Weight of dried mildly oxidized tubes was 8.1 grams.

A carbon nanotube paste was then prepared from the oxidized tubes. A 1-liter reactor was loaded with 1.010 grams of the mildly oxidized tubes and 1.820 grams of 1-pyrenemethylamine HCl (Sigma Aldrich, St. Louis, Mo.) and these were mixed thoroughly. A 1-kg bottle of chlorosulfonic acid (Sigma Aldrich, St. Louis, Mo.) was added to the reactor, and the lid was clamped on. The reactor was stoppered and equipped with overhead stirring. The stirring was set at 400 rpm and maintained for 3 days. A 4-liter jacketed quench vessel was equipped with an overhead stirrer, and a recirculating chiller set to 5° C. 1.5 liters of DI water were placed in this reactor. When the DI water reached 5° C., vacuum was used to slowly draw over the chlorosulfonic acid mixture. The temperature was kept between 10° C. and 20° C. by adjusting a release valve on the vacuum line to slow or speed the transfer rate. When this was finished, 500 mL of DI water was added to the 1-liter reactor, and this was rapidly transferred to the quench vessel. The temperature was set to 15° C., and a peristaltic pump was used to deliver 1.75 liters of 28-30% ammonium hydroxide (VWR, J.T. Baker®, Batavia, Ill.) at 7 mL/min. The resulting mixture was then filtered through an Advantec 3.0 PTFE filter (King Filtration Technologies, St. Louis, Mo.). The vacuum timer was set for 8 hours. The wet black solid was removed from the filter and mixed with a solution of 200 mL of 28-30% ammonium hydroxide in 1800 mL of DI water. The filtration and addition of ammonium hydroxide was then repeated. The resulting solid was again filtered through an Advantec 3.0 μM, PTFE filter. The black solid was then dispersed in 600 grams of 2-methyl-1,3-propanediol (Sigma Aldrich, St. Louis, Mo.). The final oxidized CNT paste contained 0.3 wt.-% oxidized CNT and 96.7 wt.-% solvent.

### Example 2

### Construction of Constant Heat Carbon Dioxide Sensor and Testing

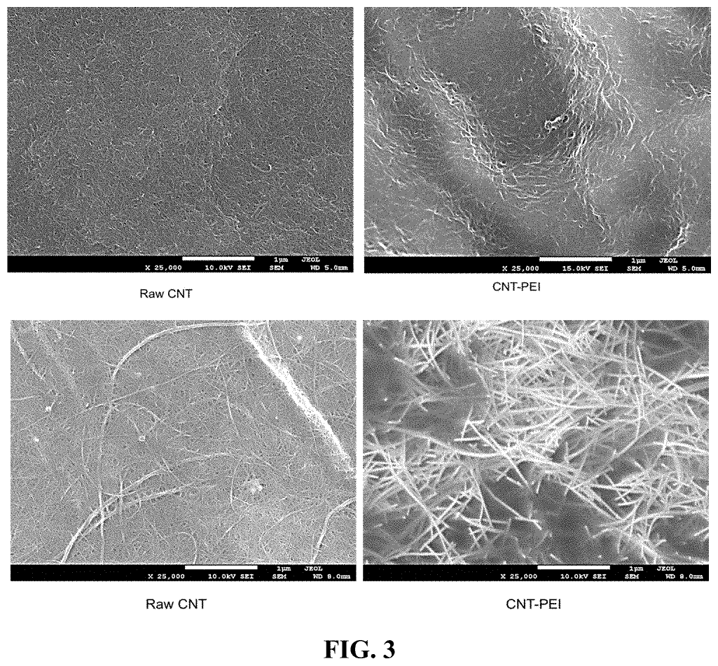

An amine-functionalized carbon nanomaterial was prepared by mixing 16.0 grams of the oxidized carbon nanotube paste prepared as described in EXAMPLE 1 and 3.0 grams of polyethylenimine (PEI, Average Mw˜800 by LS, average Mn˜600 by GPC, Sigma Aldrich, St. Louis, Mo.) using a three-roll mill (EXAKT Technologies, Inc, Oklahoma City, Okla.) at room temperature. FIG. 3 shows SEM images of raw CNTs and PEI-functionalized CNTs. As shown in FIG. 3, after the functionalization of PEI, the dimension of the CNT tubes increased, evidence PEI was bonding/wrapping on the individual CNTs.

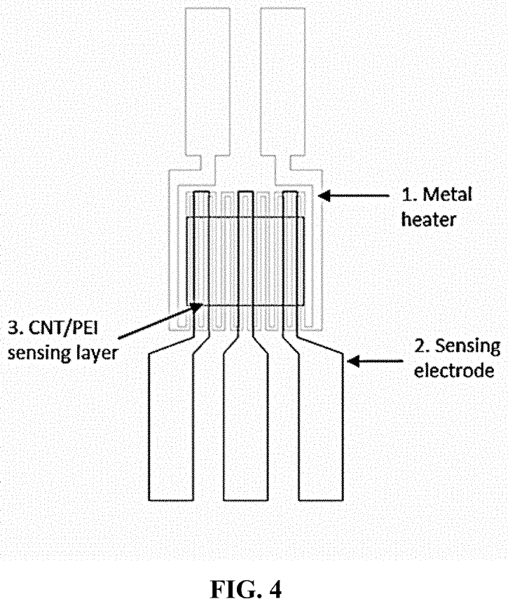

A carbon dioxide sensor was assembled according to FIG. 4 on a Kapton® substrate (CS hyde Company, Lake Villa, Ill.). The sensing electrodes were formed by screen-printing using AG-800 silver ink and were cured using Conveyor oven (HIX, Custom NP-2410, Pittsburgh, Kans.), 130° C., 45.6 inches/min, 1 pass. The active sensing layer was formed by screen printing a 1 mm×3 mm layer of the amine-functionalized carbon nanomaterial using an ATMA Electric Flat Screen Printer (Model No.: AT-60PD, Taipei, Taiwan 248), 40 mesh screen, 1 pass printing. The printed sensing film was dried in vacuum oven (StableTemp, Model 282A, Cole-Parmer, Vernon Hills, Ill., USA) at 125° C. for 5 minutes under 10 mm of Hg vacuum condition. The 5.2 mm×5.425 mm metal heater, comprised of 125-μm-wide serpentine metal traces, was made from a printable silver ink (AG-800 silver conductive ink, KAYAKU, Westborough, Mass. 01581, USA). The printed silver ink heating layer was cured using Conveyor oven (HIX, Custom NP-2410, Pittsburgh, Kans.), 130° C., 45.6 inches/min, 1 pass.

### Example 3

### Testing of Sensor Fabricated in EXAMPLE 2

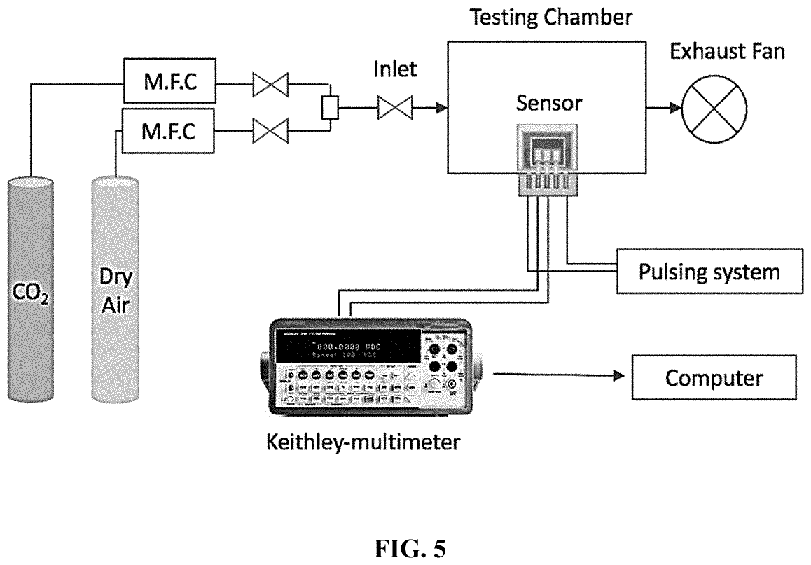

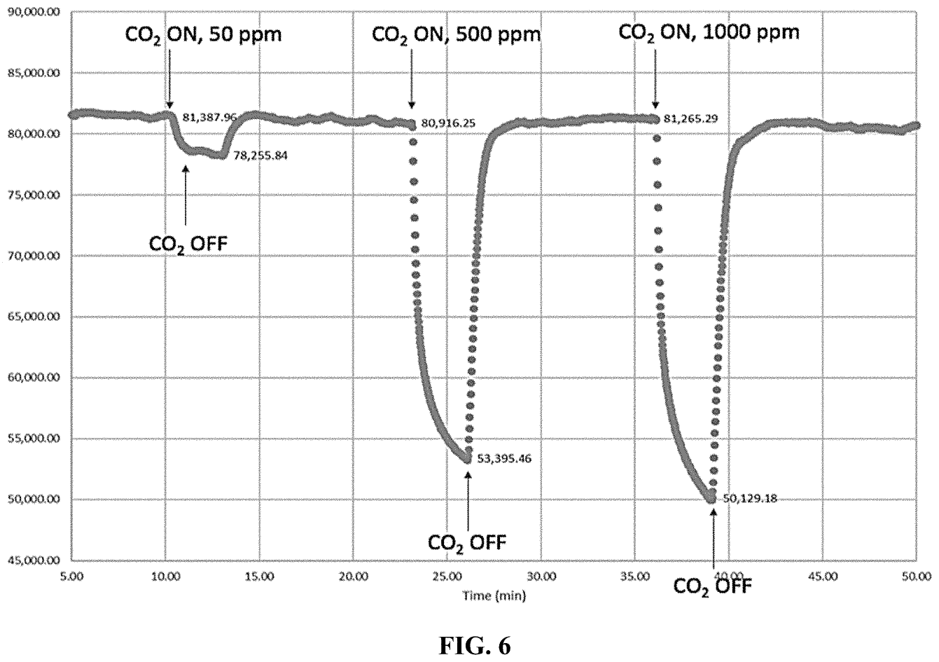

The printed CO₂ sensor fabricated in EXAMPLE 2 was connected to a testing system shown in FIG. 5. FIG. 6 shows the response of the sensor to varying carbon dioxide concentrations delivered to the testing chamber. The backside heater was set using a voltage of 3.5 V and current of 80 mA, resulting in a temperature of approximately 50°-60° C. The carrier gas for the carbon dioxide was clean dry air, and carbon dioxide concentrations of 50 ppm, 500 ppm, and 1000 ppm were used. The carrier gas was flowed over the sensor for 10 minutes, followed by 3 minutes with the carbon dioxide, and repeated two more time with various concentrations of carbon dioxide. As illustrated in FIG. 6, the sensor responded very quickly to the arrival of carbon dioxide and was easily refreshed after flow of carbon dioxide into the chamber was stopped.

### Example 4

### Pulse Heated Carbon Dioxide Sensor 1 and Testing

An amine-functionalized carbon nanomaterial was prepared by mixing 16.0 grams of a paste of oxidized carbon nanotubes prepared as described in EXAMPLE 1. 2.50 grams of polyethylenimine (Average Mw˜800 by LS, average Mn˜600 by GPC, Sigma Aldrich, St. Louis, Mo.), and 1.50 grams of 2-hydroxyethyl cellulose (Sigma Aldrich, St. Louis, Mo.) using a three-roll mill (EXAKT Technologies, Inc, Oklahoma City, Okla.) at room temperature.

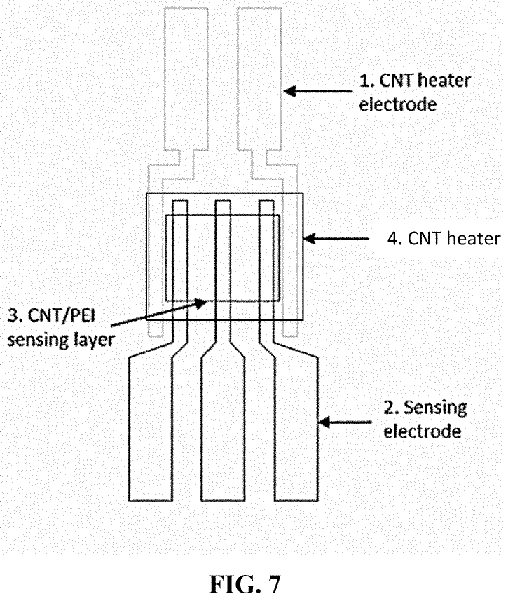

A carbon dioxide sensor was assembled according to FIG. 7 on a Kapton® substrate (CS Hyde Company, Lake Villa, Ill.) by screen printing using an ATMA Electric Flat Screen Printer (Such as ATMA screen printer, Model No.: AT-60PD, Taipei, Taiwan), 40 mesh screen, 1 pass printing. The heating layer was formed from non-oxidized carbon nanomaterial paste.

To prepare the non-oxidized carbon nanomaterial paste, a 1-liter reactor was loaded with 1.001 grams of CNTs (Zeon, Tokyo, Japan) and 1.820 grams of 1-pyrenemethylamine HCl (Sigma Aldrich, St. Louis, Mo.) and these were mixed thoroughly. A 1-kg bottle of chlorosulfonic acid (Sigma Aldrich, St. Louis, Mo.) was added to the reactor, and the lid was clamped on. The reactor was stoppered and equipped with overhead stirring. The stirring was set at 400 rpm and maintained for 3 days. A 4-liter jacketed quench vessel was equipped with an overhead stirrer, and a recirculating chiller set to 5° C. 1.5 liters of DI water were placed in this reactor. When the DI water reached 5° C., vacuum was used to slowly draw over the chlorosulfonic acid mixture. The temperature was kept between 10° C. and 20° C. by adjusting a release valve on the vacuum line to slow or speed the transfer rate. When this was finished, 500 mL of DI water was added to the 1-liter reactor, and this was rapidly transferred to the quench vessel. The temperature was set to 15° C., and a peristaltic pump was used to deliver 1.00 liters of 28-30% ammonium hydroxide (VWR, J.T. Baker®, Batavia, Ill.) at 7 mL/min. The resulting mixture was then filtered through an Advantec 3.0 μM, PTFE filter (King Filtration Technologies, St. Louis, Mo.). The vacuum timer was set for 8 hours. The wet black solid was removed from the filter and mixed with a solution of 200 mL of 28-30% ammonium hydroxide in 1800 mL of DI water. The filtration and addition of ammonium hydroxide was then repeated. The resulting solid was again filtered through an Advantec 3.0 μM, PTFE filter. The black solid was then dispersed in 600 grams of 2-methyl-1,3-propanediol (Sigma Aldrich, St. Louis, Mo.).

CNT heater electrodes were printed using AG-800 silver, and were cured at 130° C. at 45.6″/min conveyor speed at HIX oven as in EXAMPLE 2. A 4.2 mm×4.5 mm heating layer of non-oxidized CNT ink was printed between the silver electrodes and was cured in HIX oven at 70 C at 5″/min speed one time followed by an additional cure of 130 C at 45.5″/min belt speed 3 times in the same HIX oven. Sensing layer electrodes were printed on the other side of the Kapton® substrate, aligned with heater elements using AG-800 silver, and were cured the same way as the heater electrodes. A 1 mm×3 mm layer of the CO₂ sensing amine-functionalized carbon nanomaterial was printed between the sensing layer electrodes. They were dried in HIX oven at 130° C. at 45.5″/min belt speed 1 time, and were cured in vacuum oven (StableTemp, Model 282A, Cole-Parmer, Vernon Hills, Ill., USA) at 125° C. for 5 minutes under 10 mm of Hg vacuum condition.

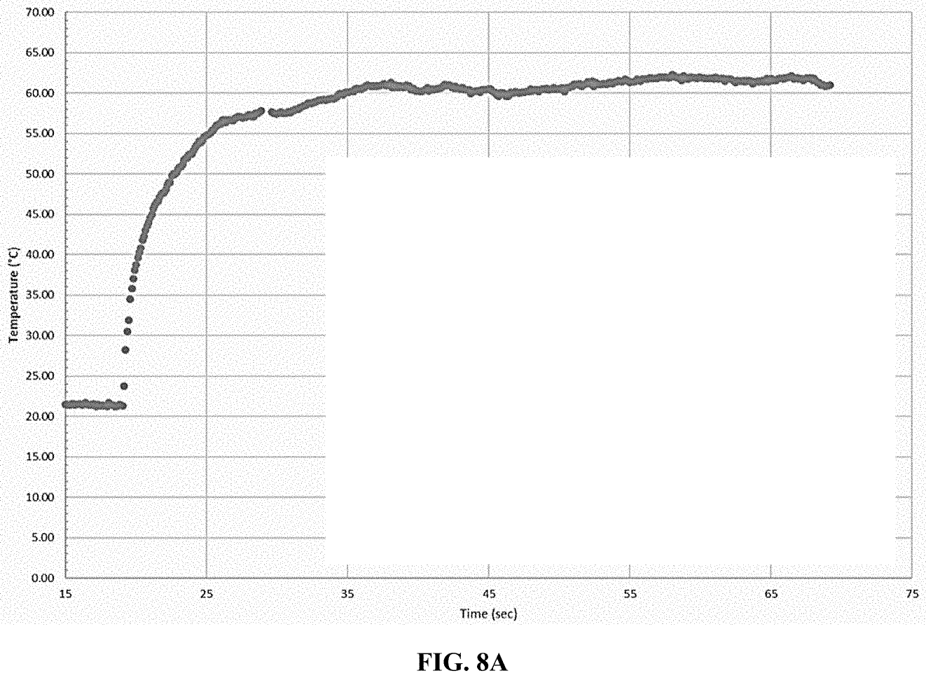

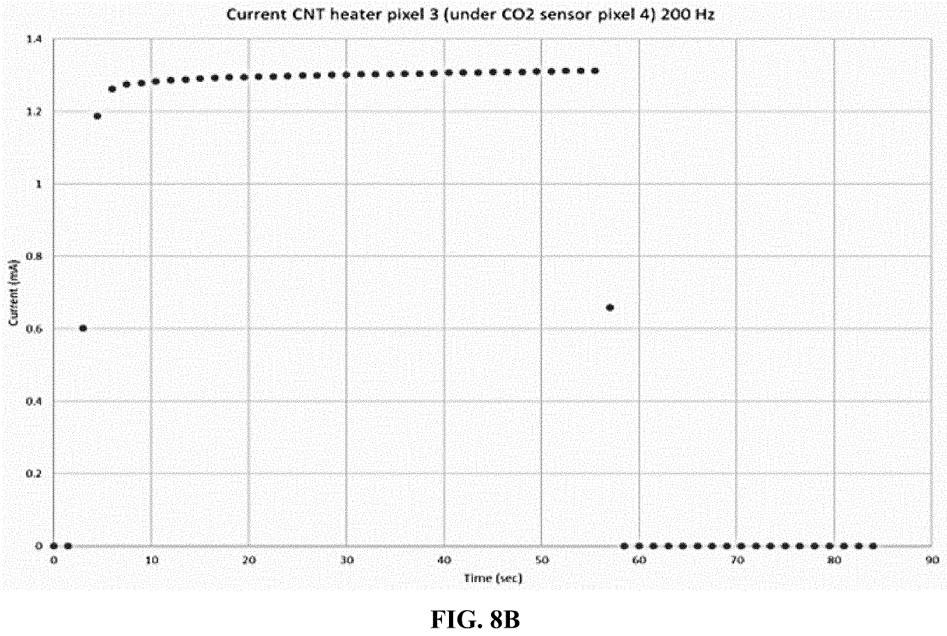

FIG. 8A shows the surface temperature of the sensor measured using an IR camera (FLIR Ax5 series, FLIR, Nashua, N.H.). The temperature of the sensor was set by pulsing the sensor electrodes using 50 V AC pulses of 200 μs pulse width at 200 Hz frequency with varying pulse power and current. A stable temperature of approximately 62° C. was held by using a current of 1.3 mA, shown in FIG. 8B.

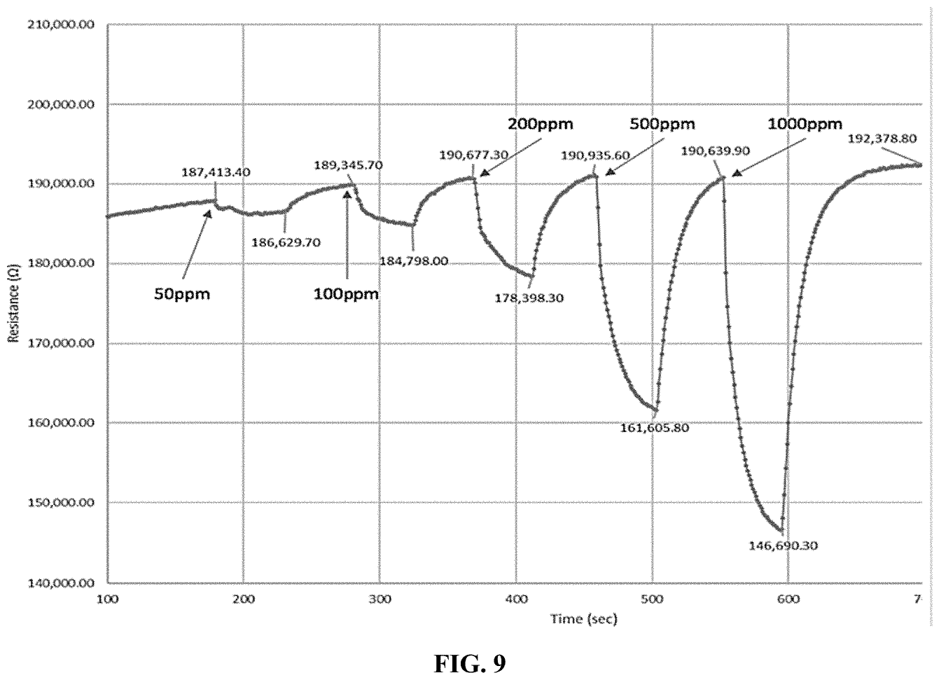

FIG. 9 shows the response of the sensor to varying carbon dioxide concentrations. The temperature of the sensor was set pulsing the sensor at 200 Hz, with 50 V AC pulses of 200 μs pulse at 1.3 mA pulse current, resulting in a temperature of approximately 50°-60° C. The carrier gas for the carbon dioxide was clean dry air, and carbon dioxide concentrations of 50 ppm, 500 ppm, and 1000 ppm were used. The carrier gas was flushed twice, and then flowed over the sensor for 60 seconds, followed by 60 seconds with the carbon dioxide, and repeated two more time with various concentrations of carbon dioxide. Again, the sensor responded very quickly to the arrival of carbon dioxide and was easily refreshed after flow of carbon dioxide into the chamber was stopped.

### Example 5

### Pulse Heated Carbon Dioxide Sensor 2 and Testing

An amine-functionalized carbon nanomaterial was prepared by mixing 16.0 grams of oxidized carbon nanotube paste as prepared in EXAMPLE 1 and 3.0 grams of a 50% PEI solution in water (Average Mw˜25,000, Sigma Aldrich, St. Louis, Mo.) using a three-roll mill (EXAKT Technologies, Inc, Oklahoma City, Okla.) at room temperature.

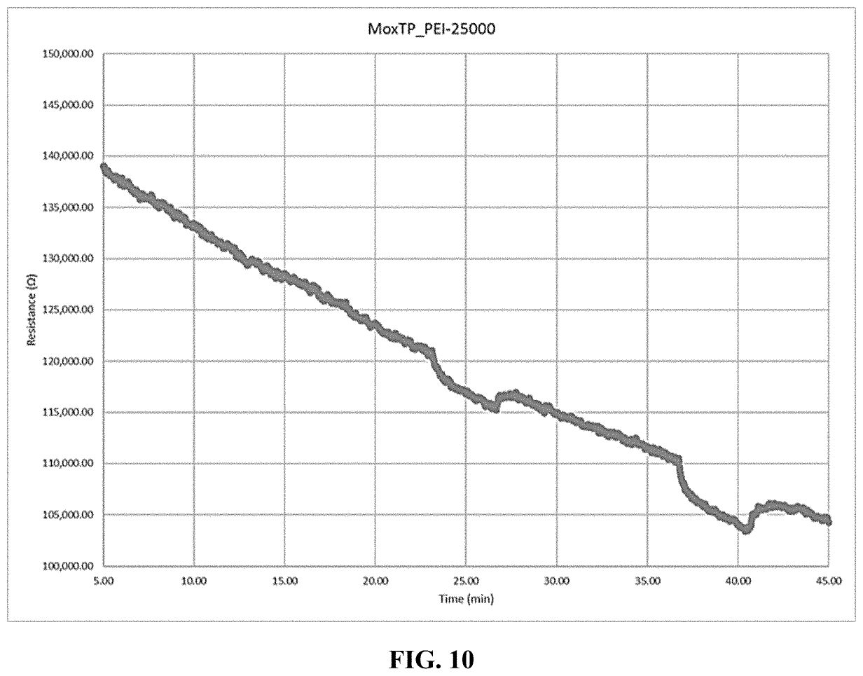

A sensor was fabricated and tested using the same parameters shown in EXAMPLE 4. As shown in FIG. 10, the responsiveness was not as responsive to carbon dioxide as sensors fabricated using lower-molecular-weight PEI.

### Example 6

### Pulse Heated Carbon Dioxide Sensor 3 and Testing

An amine-functionalized carbon nanomaterial was prepared by mixing 16.0 grams of oxidized carbon nanotube paste as prepared in EXAMPLE 1, 2.50 grams of a 50% PEI solution in water (Average Mw˜25,000, Sigma Aldrich, St. Louis, Mo.), and 1.50 grams of 2-hydroxyethyl cellulose using a three-roll mill (EXAKT Technologies, Inc, Oklahoma City, Okla.) at room temperature. The 2-hydroxyethyl cellulose was used to trap moisture to attempt to amplify the sensing mechanism.

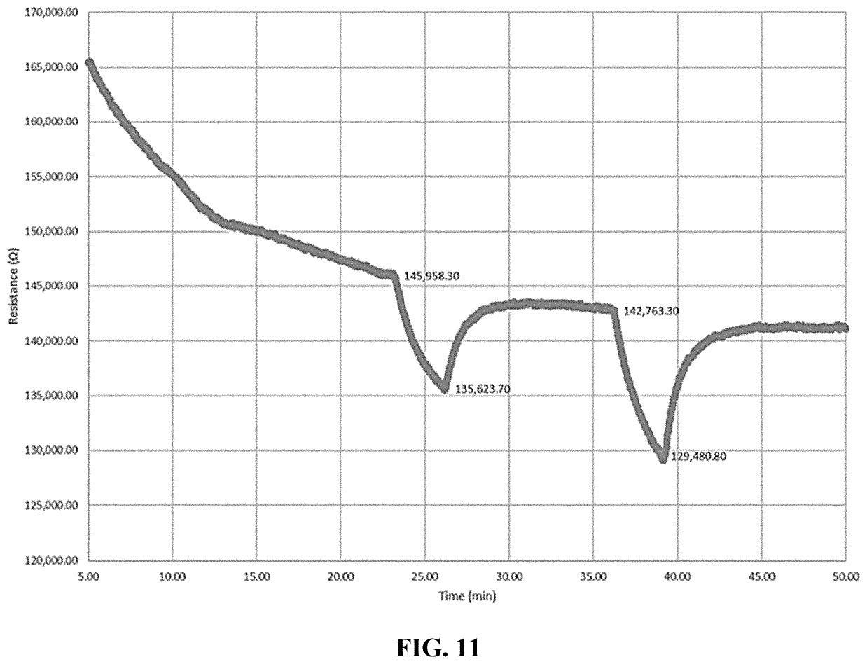

A sensor was fabricated and tested using the same parameters shown in EXAMPLE 4. FIG. 11 shows the test result. While the addition of the 2-hydroxyethyl cellulose did improve the signal compared to Example 5, this method may cause drifting problems in the final sensor.

### Example 7

### Amine-Functionalized Carbon Nanotube Material

An amine-functionalized carbon nanomaterial was prepared by mixing 16.0 grams of oxidized carbon nanotube paste as prepared in EXAMPLE 1 and 1.78 grams of PEI solution (average Mw˜800 by LS, average Mn˜600 by GPC, Sigma Aldrich, St. Louis, Mo.), using a three-roll mill (EXAKT Technologies, Inc, Oklahoma City, Okla.) at room temperature. The three-roll mill processing parameters are shown in Table 1.

TABLE 1
Three-roll mill parameters

| | Front | Back | | | |
|---|---|---|---|---|---|
| | gap | gap | Speed | Processing | |
| Steps | (μm) | (μm) | (rpm) | time (min) | Processing/Observation |
| 1 | 50 | 50 | 30 | 1~2 | Add oxidized CNT paste into 3-roll mill |
| 2 | 50 | 50 | 30 | 1~2 | Add PEI solution into 3-roll mill |
| 3 | 20 | 20 | 100 | 2 | Obvious viscosity increase |
| 4 | 10 | 10 | 300 | 2 | Paste became uniform |
| 5 | 5 | 5 | 300 | 10~15 | Collect processed paste at the end of this step |

### Example 8

### Fabrication of a Pulse-Heated 4-Pixel Carbon Dioxide Sensor

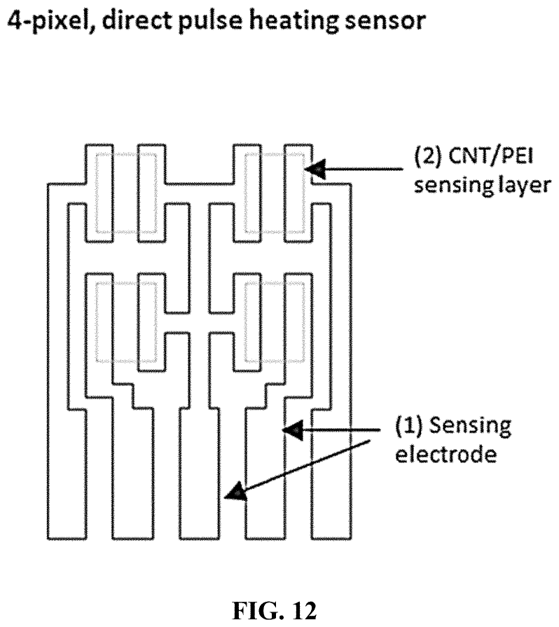

A 4-pixel pulse-heated sensor structure was fabricated according to FIG. 12. The substrate was 5-mil-thick Kapton® film (HN type) from Dupont. Sensing electrodes were printed using AG-800 silver, and were cured at 130 C at 45.6″/min conveyor speed at HIX oven as in EXAMPLE 2. Four 1 mm×3 mm layers of the CO₂ sensing ink from EXAMPLE 7 were then printed between the electrodes as shown in FIG. 12. The sensing ink was dried in a HIX oven at 130° C. at 45.5″/min belt speed 1 time, and was cured in a vacuum oven (StableTemp, Model 282A, Cole Parmer, Vernon Hills, Ill.) at 125° C. for 5 minutes under 10 mm of Hg vacuum condition.

### Example 9

### Direct Pulse Heating Temperature Output

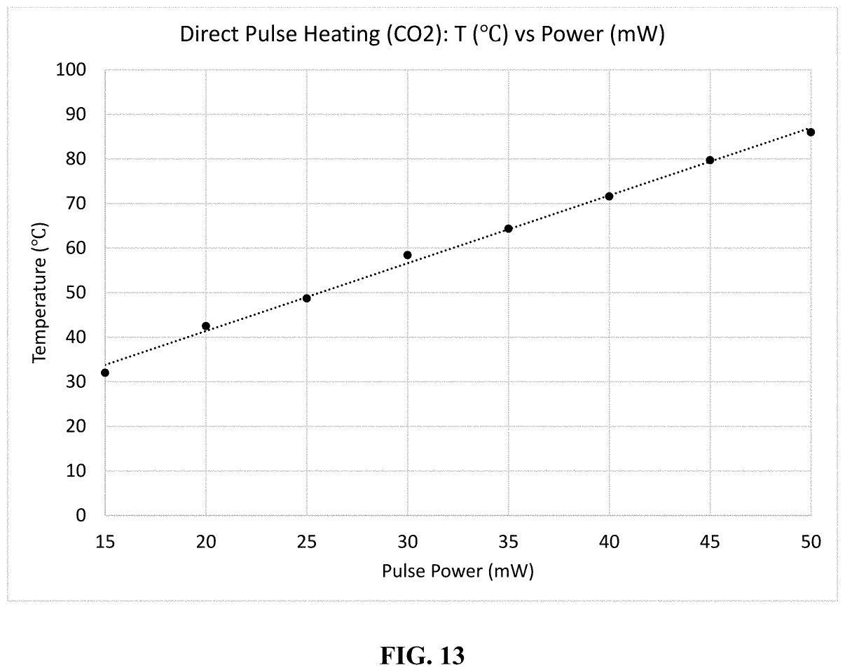

In this example, a CO₂ sensor as fabricated in EXAMPLE 8 was connected to internally fabricated pulse heating electronics. The heating electronics contained a DC converter capable of generating 50 V DC power and used power MOSFETs at the DC converter's output to switch the 50 V delivered to each sensor. When the MOSFET was switched on, the DC converter's 50 V output is delivered to the respective sensor. But when the MOSFET is switched off, the DC converter's 50 V to the sensor is cut off, thus producing 50 V rectangular wave pulses at the sensor. The on/off state of the power MOSFETs was controlled by a microcontroller. The sensor electrodes were pulsed with a pulse voltage of 50 V, pulse width of 200 μs. Pulse frequency was automatically varied to achieve the desired pulse power. An example of temperature output vs. pulse power is shown in FIG. 13.

### Example 10

### Separate Heater Temperature Output

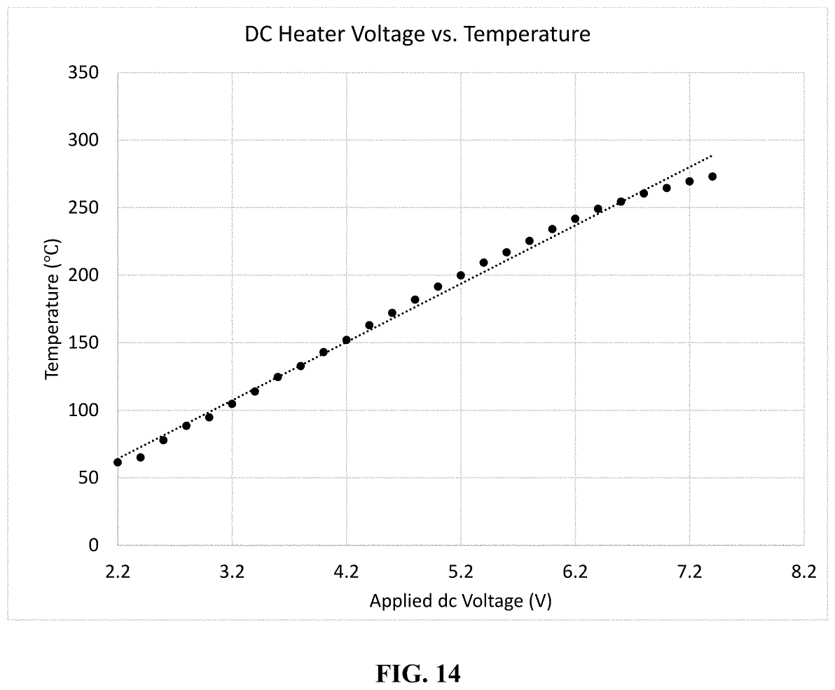

In this example, a continuous DC voltage was applied to a sensor fabricated in EXAMPLE 2 using a Keithley 2230-301-1 triple-channel DC power supply. An example of the temperature output of the backside DC silver heater as a function of applied DC voltage to the heater is shown below FIG. 14.

### Example 11

### Sensing Using Direct Pulse Heating

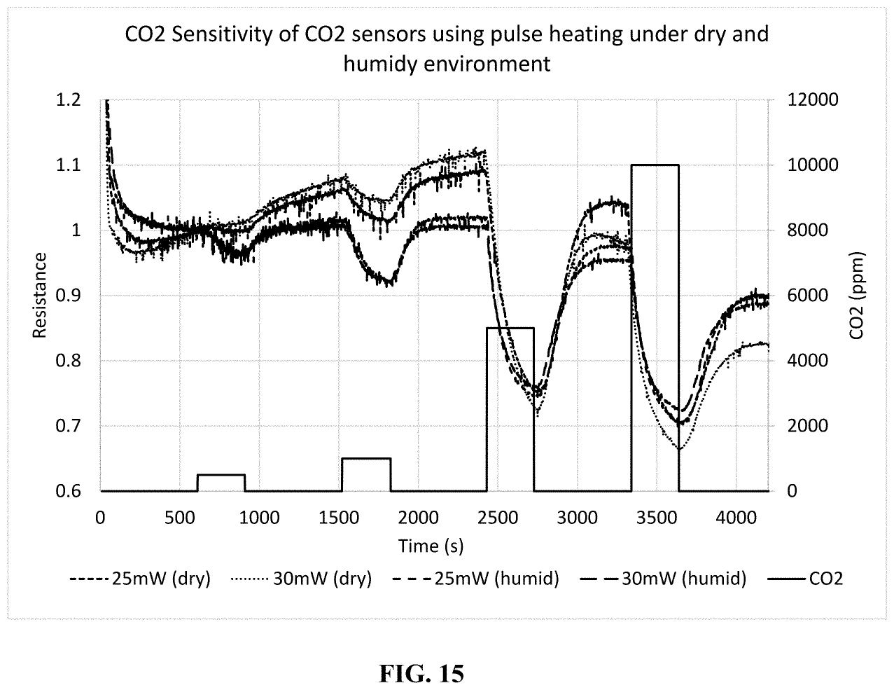

Direct pulse-heated 4-pixel sensors prepared in EXAMPLE 8 were tested using the pulse heating electronics described in EXAMPLE 9 at 25-30 mW pulse power with a pulse voltage of 50 V and pulse width of 200 μs to achieve a temperature of approximately 50-60° C. under dry (0%-3% RH) and high humidity conditions (80% RH). The resistance of the sensors was measured using a Flukemeter 8845A digital multimeter. Real time data under dry and humid conditions at 500, 1000, 5,000, and 10,000 ppm concentrations of CO₂ is shown in FIG. 15. The sensors show a good concentration dependency. CO₂ sensing at 500 ppm is <1% for dry conditions and 4-5% at high humidity.

### Example 12

### CO₂ Sensing Using Backside Silver DC Heater

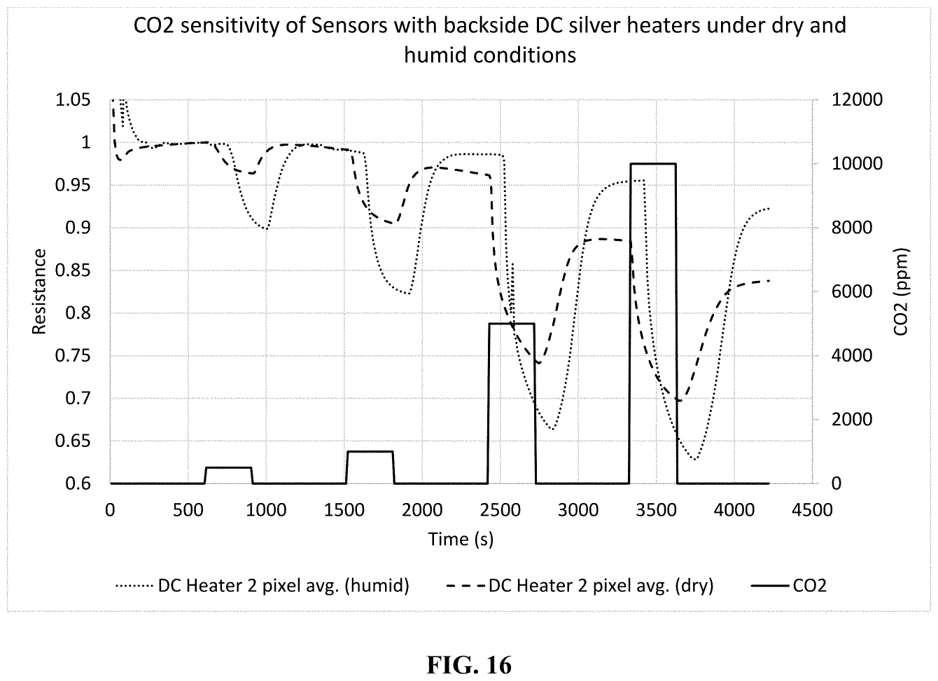

Backside DC heated 2-pixel CO₂ sensors prepared in EXAMPLE 2 were tested with 2.2 V DC voltage applied to the heater to reach a temperature of approximately 60° C. Real time data for 500, 1000, 5,000, and 10,000 ppm of CO₂ under dry and humid conditions is shown in FIG. 16. The sensor shows the concentration dependency of the sensor. On average, backside-heated CO₂ sensors saw a 47% increase in sensitivity under high humidity conditions. CO₂ sensitivity response for backside heaters is 2-3% for 500 ppm and under dry conditions (0-3% humidity) and 8-11% for high humidity conditions (80% humidity tested). The response is shown in FIG. 16.

### Example 13

### 4-Pixel CO₂ Sensor Selectivity

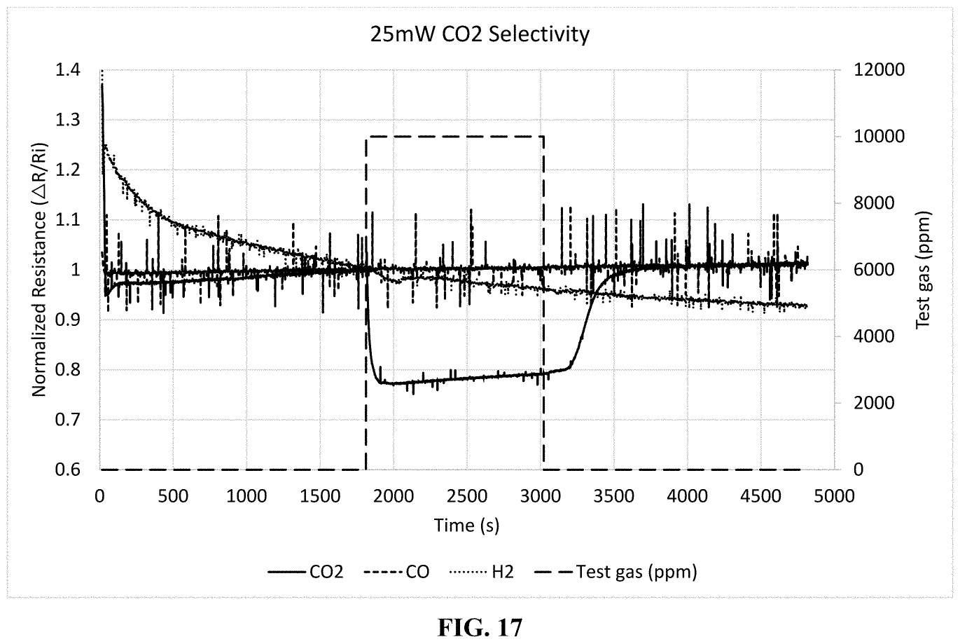

4-pixel CO₂ sensors prepared in EXAMPLE 8 were tested for selectivity for CO₂ over other gases such as CO, H₂, and CH₄. The CO₂ sensor electrodes were pulse heated at 25 mW power at a pulse voltage of 50 V and pulse width of 200 us to achieve a temperature of about 50° C., and was exposed to 10,000 ppm concentration of CO₂, CO, and H₂ in dry condition separately. The sensors showed a very good response to CO₂ while showing no response to other gas analytes, indicating a good selectivity toward CO₂. The response is shown in FIG. 17.

1. A carbon dioxide sensor comprising:
first and second sensing electrodes formed from electrically conductive material, the sensing electrodes spaced apart from one another and positioned on a substrate; and
an active sensing layer formed from polyethyleneimine-functionalized carbon nanomaterials comprising branched polyethyleneimine polymers having a weight average molecular weight of less than about 25,000, the active sensing layer being positioned on the substrate in direct physical contact with at least a portion of the first and second sensing electrodes and being positioned to detect a change in an electrical characteristic in response to a change in carbon dioxide concentration in proximity to the active sensing layer.

2. The carbon dioxide sensor of claim 1, wherein the branched polyethyleneimine polymers have a weight average molecular weight of less than about 1,000.

3. The carbon dioxide sensor of claim 1, wherein the active sensing layer is formed by dispensing a composition onto the substrate, the composition comprising the polyethyleneimine-functionalized carbon nanomaterials dispersed in a liquid medium.

4. The carbon dioxide sensor of claim 3, wherein the liquid medium comprises a solvent selected from the group consisting of water, alcohols, diols, polar water-miscible solvents, and combinations thereof.

5. The carbon dioxide sensor of claim 1, wherein the polyethyleneimine-functionalized carbon nanomaterials comprise polyethyleneimine polymers chemically bonded to oxidized carbon nanotubes.

6. The carbon dioxide sensor of claim 1, further comprising a heating layer formed on an opposite side of the substrate from the active sensing layer for maintaining or changing the temperature of the active sensing layer during operation of the carbon dioxide sensor.

7. The carbon dioxide sensor of claim 6, further comprising first and second heating electrodes formed from electrically conductive material, each of the heating electrodes being in direct physical contact with the heating layer for providing a series of electrical pulses to the heating layer during operation of the sensor.

8. The carbon dioxide sensor of claim 1, wherein the substrate comprises an organic polymer selected from the group consisting of polyimides, polyamides, polysulfones, poly ether sulfones, polyether ether ketone (PEEK), polyethylene terpthalate (PET), polytetrafluoroethylene (PTFE), acrylates, methacrylates, styrenics, cycloolefin polymers, cycloolefin copolymers, polyesters, polyethylene naphthalates, and combinations thereof.

9. A sensor array comprising the carbon dioxide sensor according to claim 1, wherein the first and second sensing electrodes are positioned to detect a change in a first portion of the active sensing layer, the sensor array further comprising a third sensing electrode positioned to detect a change in an electrical characteristic in response to a change in an analyte concentration in proximity to at least one of: (a) a second portion of the active sensing layer different from said first portion of the active sensing layer; and (b) a second active sensing layer.

10. A method of determining carbon dioxide concentration of a target gas with the sensor according to claim 1, the method comprising:
(i) contacting the active sensing layer with the target gas; and
(ii) measuring an electrical characteristic between the at least first and second electrodes.

11. The method of claim 10, further comprising heating the active sensing layer to an operational temperature of from about 25° C. to about 85° C.

12. The method of claim 11, wherein the sensor further comprises a heating layer and at least first and second heating electrodes formed on an opposite side of the substrate from the active sensing layer, and wherein the heating step comprises providing a series of electrical pulses from the heating electrodes to the heating layer.

13. A method of forming a carbon dioxide sensor, the method comprising dispensing a composition onto a substrate, the composition comprising polyethyleneimine-functionalized carbon nanomaterials dispersed in a liquid medium, the polyethyleneimine-functionalized carbon nanomaterials comprising branched polyethyleneimine polymers having a weight average molecular weight of less than about 25,000.

14. The method of claim 13, wherein the branched polyethyleneimine polymers have a weight average molecular weight of less than about 1,000.

15. The method of claim 13, wherein the substrate comprises an organic polymer selected from the group consisting of polyimides, polyamides, polysulfones, poly ether sulfones, polyether ether ketone (PEEK), polyethylene terpthalate (PET), polytetrafluoroethylene (PTFE), acrylates, methacrylates, styrenics, cycloolefin polymers, cycloolefin copolymers, polyesters, polyethylene naphthalates, and combinations thereof.

16. The method of claim 13, wherein liquid medium comprises a solvent selected from the group consisting of water, alcohols, diols, polar water-miscible solvents, and combinations thereof.

17. The method of claim 13, wherein the polyethyleneimine-functionalized carbon nanomaterials comprise polyethyleneimine polymers chemically bonded to oxidized carbon nanotubes.

18. A composition comprising polyethyleneimine-functionalized carbon nanomaterials dispersed in a liquid medium, the polyethyleneimine-functionalized carbon nanomaterials comprising branched polyethyleneimine polymers having a weight average molecular weight of less than about 25,000.

19. The composition of claim 18, wherein the branched polyethyleneimine polymers have a weight average molecular weight of less than about 1,000.

20. The composition of claim 18, wherein the polyethyleneimine-functionalized carbon nanomaterials comprise polyethyleneimine polymers chemically bonded to oxidized carbon nanotubes.
